SEQUENCE LISTING (20 sequences)

SEQ ID NO: 1 - DNA, 20 bp, synthetic construct
  source 1..20: mol_type = other DNA
ttcggctcacgctgggttgt

SEQ ID NO: 2 - DNA, 20 bp, synthetic construct
  source 1..20: mol_type = other DNA
cgaggttttgttccgaagcc

SEQ ID NO: 3 - DNA, 20 bp, synthetic construct
  source 1..20: mol_type = other DNA
gggtaatgtgacgaggtttt

SEQ ID NO: 4 - DNA, 20 bp, synthetic construct
  source 1..20: mol_type = other DNA
ggggtaatgtgacgaggttt

SEQ ID NO: 5 - DNA, 20 bp, synthetic construct
  source 1..20: mol_type = other DNA
cacacctgatcaaatttctc

SEQ ID NO: 6 - DNA, 20 bp, synthetic construct
  source 1..20: mol_type = other DNA
gcacacctgatcaaatttct

SEQ ID NO: 7 - DNA, 20 bp, synthetic construct
  source 1..20: mol_type = other DNA
ctgctcagtcttcagaaaat

SEQ ID NO: 8 - DNA, 20 bp, synthetic construct
  source 1..20: mol_type = other DNA
tctgctcagtcttcagaaaa

SEQ ID NO: 9 - DNA, 20 bp, synthetic construct
  source 1..20: mol_type = other DNA
aagtttaagagggtctgtgt

SEQ ID NO: 10 - DNA, 20 bp, synthetic construct
  source 1..20: mol_type = other DNA
tgttggcagaggatggtcaa

SEQ ID NO: 11 - DNA, 20 bp, synthetic construct
  source 1..20: mol_type = other DNA
actgttggcagaggatggtc

SEQ ID NO: 12 - DNA, 20 bp, synthetic construct
  source 1..20: mol_type = other DNA
tttggggattgtgctctcat

SEQ ID NO: 13 - DNA, 20 bp, synthetic construct
  source 1..20: mol_type = other DNA
ggttctgatgtttgtagtgt

SEQ ID NO: 14 - DNA, 20 bp, synthetic construct
  source 1..20: mol_type = other DNA
tggttctgatgtttgtagtg

SEQ ID NO: 15 - DNA, 20 bp, synthetic construct
  source 1..20: mol_type = other DNA
gggcttggttctgatgtttg

SEQ ID NO: 16 - DNA, 20 bp, synthetic construct
  source 1..20: mol_type = other DNA
cgggcttggttctgatgttt

SEQ ID NO: 17 - DNA, 20 bp, synthetic construct
  source 1..20: mol_type = other DNA
tggggtcatatgttcctggc

SEQ ID NO: 18 - DNA, 20 bp, synthetic construct
  source 1..20: mol_type = other DNA
ttggggtcatatgttcctgg

SEQ ID NO: 19 - DNA, 20 bp, synthetic construct
  source 1..20: mol_type = other DNA
ggtttcagtttagtggaagc

SEQ ID NO: 20 - DNA, 20 bp, synthetic construct
  source 1..20: mol_type = other DNA
tgaattgaagcgtgtgagtc

(19) United States
(12) Patent Application Publication
(10) Pub. No.: US 2026/0166073 A1
(43) Pub. Date: Jun. 18, 2026

(54) MTOR THERAPEUTICS FOR CANCER

(71) Applicant: GMP BIOTECHNOLOGY LIMITED, Hong Kong (CN)

(72) Inventor: Vuong TRIEU, Agoura Hills, CA (US)

(21) Appl. No.: 19/290,587

(22) Filed: Aug. 5, 2025

Related U.S. Application Data
(63) Continuation of application No. PCT/US2024/014520, filed on Feb. 5, 2024.
(60) Provisional application No. 63/443,659, filed on Feb. 6, 2023.

(51) Int. Cl. A61K 31/7125; A61K 31/167; A61K 31/501; A61K 31/7115; A61K 31/712; A61K 45/06; A61P 35/00; G01N 33/50; G01N 33/68

(52) U.S. Cl. CPC A61K 31/7125; A61K 31/167; A61K 31/501; A61K 31/7115; A61K 31/712; A61K 45/06; A61P 35/00; G01N 33/5005; G01N 33/6884; G01N 33/689

(57) ABSTRACT

This invention relates to methods, agents and uses for treating cancer. More particularly, this invention discloses methods, agents and uses for inhibiting and/or suppressing mTOR, which can provide improved clinical outcomes for cancer. This invention provides stable formulations of new mTOR agents, including antisense oligonucleotide structures, as well as combinations with other agents and therapies for treating cancer. This invention includes aspects for selecting subjects or sub-populations who benefit from mTOR agents against cancer.

### SEQUENCE LISTING

This application includes a sequence listing submitted electronically as an ST.26 file created on Jan. 26, 2024, named 018988-009US1_SL.xml, which is 18,396 bytes in size.

### TECHNICAL FIELD

This invention relates to methods, agents and uses for treating cancer. More particularly, this invention discloses methods, agents and uses for inhibiting or suppressing mTOR, which provide improved clinical outcomes for cancer. This invention provides stable formulations of new mTOR agents, including antisense oligonucleotide compositions, as well as combinations with other therapies for treating cancer. This invention includes biomarker methods for selecting subjects who would benefit from use of mTOR inhibitor agents and therapies.

### BACKGROUND

Activity of mTOR has been found to be dysregulated in some types of cancer including breast, prostate, lung, melanoma, bladder, and brain carcinomas. In some cases, mTOR may be abnormally activated which can increase tumor growth and metastasis. Over-activation of mTOR signaling has been suspected in the initiation and development of tumors. In addition, overexpression of downstream mTOR effectors correlates with poor cancer prognosis.

Some cancer therapies have been developed using mTOR inhibitors which are natural products of a streptomyces species or derivatives of a natural macrocyclic lactone. The therapeutic strategy was to reduce overexpression of mTOR complexes in tumors.

However, drawbacks and complications of mTOR inhibitors include complex feedback loops which can cause mTOR inhibitors to lose efficacy and/or promote cancer.

In other cases, immune checkpoint inhibitors are greatly advancing therapies in oncology and have become the first or second line of treatment for many cancers. However, drawbacks and complications of immune checkpoint inhibitors include lack of efficacy in over half of some cancer patients.

What is needed are new agents and methods for inhibiting mTOR. New structures and compositions for inhibiting mTOR can provide novel therapeutic methods. Further, combination with known mTOR inhibitors can supply new therapies.

There is an urgent need for methods to select subjects who would benefit from use of mTOR inhibitor agents and therapies. What is needed are methods using biomarkers to identify those who would benefit from mTOR therapy.

### BRIEF SUMMARY

This invention relates to methods, agents and uses of mTOR therapy treating cancer.

This invention includes new agents and methods for inhibiting mTOR. The novel structures and compositions of this disclosure for inhibiting mTOR can provide novel therapeutic methods.

In some embodiments, new therapies can be supplied by a combination of novel mTOR-suppressing agents of this disclosure with additional mTOR inhibitor agents.

This invention includes methods to select subjects who would benefit from use of mTOR agents and mTOR therapies. A range of specific biomarkers can be used to identify those who would benefit from mTOR therapy.

Embodiments of this invention include the following:

A method for treating or ameliorating a symptom of cancer in a human or animal subject in need, the method comprising administering a therapeutically sufficient amount of a pharmaceutical composition comprising an agent for suppressing mTOR to the subject.

An agent for suppressing mTOR for treating or ameliorating a symptom of cancer in a human or animal subject in need.

Use of a composition comprising an agent for suppressing mTOR in the preparation of a medicament for treating or ameliorating a symptom of cancer in a human or animal subject in need.

The method, agent or use above, wherein the cancer is a sarcoma, a lung cancer, an ovarian cancer, or a pancreatic cancer.

The method, agent or use above, comprising using one or more biomarkers to select subjects who benefit from the method, agent or use.

The method, agent or use above, wherein the one or more biomarkers are a level of a tumor mutation burden (TMB), a tumor neoantigen, a tumor-associated immune cell, or a combination thereof.

The method, agent or use above, wherein the one or more biomarkers are a level of a basophil cell, a B-cell, a T-cell, a T-helper cell (Th), an eosinophil cell, a macrophage cell, a mesenchymal stem cell, or a combination thereof, as determined in a tumor microenvironment.

The method, agent or use above, wherein the one or more biomarkers are a level of a CD4+ cell, a memory T-cell, a CD8+ cell, a natural killer T-cell, a regulatory T-cell, a type 1 T-helper cell (Th1), a type 2 T-helper cell (Th2), or a combination thereof, as determined in a tumor microenvironment.

The method, agent or use above, wherein the cancer is a sarcoma and the one or more biomarkers are a level of a tumor mutation burden (TMB), a tumor neoantigen level, a mesenchymal stem cell determined in a tumor microenvironment, a type 1 T-helper cell (Th) determined in a tumor microenvironment, or a combination thereof.

The method, agent or use above, wherein the cancer is a lung squamous cell carcinoma and the one or more biomarkers are a level of a CD4+ cell determined in a tumor microenvironment, a memory T-cell determined in a tumor microenvironment, an eosinophil cell determined in a tumor microenvironment, or a combination thereof.

The method, agent or use above, wherein the cancer is a pancreatic cancer and the one or more biomarkers are a level of a CD8+ cell determined in a tumor microenvironment, a tumor mutation burden (TMB), or a combination thereof.

The method, agent or use above, wherein the cancer is an ovarian cancer and the one or more biomarkers are a level of a natural killer T-cell determined in a tumor microenvironment, a tumor neoantigen determined in a tumor microenvironment, or a combination thereof.

The method, agent or use above, wherein the agent for suppressing or inhibiting mTOR is an mTOR-specific antisense oligonucleotide.

The method, agent or use above, wherein the agent for suppressing mTOR is an mTOR-specific antisense oligonucleotide complementary to a mTOR transcript and 15-30 nucleotides in length, or complementary to a mTOR pre-RNA, pre-mRNA or mRNA and 18-21 nucleotides in length.

The method, agent or use above, wherein the agent for suppressing or inhibiting mTOR is an mTOR-specific antisense oligonucleotide as follows: Table 1 and chemically-modified variants thereof, LNA variants thereof, gapmer variants thereof, and any combination or pooling thereof.

The agent, use or method above, wherein the mTOR-specific antisense oligonucleotides have no more than one or two mismatches as compared to a target human mTOR.

The agent, use or method above, wherein the mTOR-specific antisense oligonucleotides reduce a mTOR transcript level by at least 60%, or at least 70%, or at least 80%, or at least 90%.

The agent, use or method above, wherein the mTOR-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The agent, use or method above, wherein the antisense oligonucleotide is conjugated to a polyethylene glycol, a lipid, or a triantennary N-acetyl-galactosamine.

The method, agent or use above, comprising antisense oligonucleotide in a carrier of sterile water for injection, saline, isotonic saline, or a combination thereof.

The method, agent or use above, wherein the antisense oligonucleotides are substantially free of excipients.

The method, agent or use above, wherein the antisense oligonucleotides are stable for at least 14 days in carrier at 37° C.

The method, agent or use above, wherein the administration or use of the composition is combined with a standard of care treatment for the cancer, wherein the standard of care treatment comprises chemotherapy or radiation therapy, and wherein the administration or use of the composition is performed in the absence of immune checkpoint inhibitors.

The method, agent or use above, wherein the administration or use decreases mortality rate at month 6, 12, 18, 24, 30, or 36.

The method, agent or use above, wherein the administration or use increases survival rate at month 6, 12, 18, 24, 30, or 36.

A method for treating or ameliorating a symptom of cancer in a human or animal subject in need, the method comprising: administering a therapeutically sufficient amount of a pharmaceutical composition comprising an mTOR-specific antisense oligonucleotide in combination with an agent for inhibiting mTOR to the subject.

The method above, wherein the cancer is a sarcoma, a lung cancer, an ovarian cancer, or a pancreatic cancer.

The method above, comprising using one or more biomarkers to select subjects who benefit from the method.

The method above, wherein the one or more biomarkers are a level of a tumor mutation burden (TMB), a tumor neoantigen, a tumor-associated immune cell, or a combination thereof.

The method above, wherein the one or more biomarkers are a level of a basophil cell determined in a tumor microenvironment, a B-cell determined in a tumor microenvironment, a T-cell determined in a tumor microenvironment, a T-helper cell (Th) determined in a tumor microenvironment, an eosinophil cell determined in a tumor microenvironment, a macrophage cell determined in a tumor microenvironment, a mesenchymal stem cell determined in a tumor microenvironment, or a combination thereof.

The method above, wherein the one or more biomarkers are a level of a CD4+ cell determined in a tumor microenvironment, a memory T-cell determined in a tumor microenvironment, a CD8+ cell determined in a tumor microenvironment, a natural killer T-cell determined in a tumor microenvironment, a regulatory T-cell determined in a tumor microenvironment, a type 1 T-helper cell (Th1) determined in a tumor microenvironment, a type 2 T-helper cell (Th2) determined in a tumor microenvironment, or a combination thereof.

The method above, wherein the cancer is a sarcoma and the one or more biomarkers are a level of a tumor mutation burden (TMB), a tumor neoantigen level, a mesenchymal stem cell determined in a tumor microenvironment, a type 1 T-helper cell (Th) determined in a tumor microenvironment, or a combination thereof.

The method above, wherein the cancer is a lung squamous cell carcinoma and the one or more biomarkers are a level of a CD4+ cell determined in a tumor microenvironment, a memory T-cell determined in a tumor microenvironment, an eosinophil cell determined in a tumor microenvironment, or a combination thereof.

The method above, wherein the cancer is a pancreatic cancer and the one or more biomarkers are a level of a CD8+ cell determined in a tumor microenvironment, a tumor mutation burden (TMB), or a combination thereof.

The method above, wherein the cancer is an ovarian cancer and the one or more biomarkers are a level of a natural killer T-cell determined in a tumor microenvironment, a tumor neoantigen determined in a tumor microenvironment, or a combination thereof.

The method above, wherein the mTOR-specific antisense oligonucleotide is any of the following: Table 1 and chemically-modified variants thereof, LNA variants thereof, gapmer variants thereof, and any combination or pooling thereof.

The method above, wherein the mTOR-specific antisense oligonucleotides have no more than one or two mismatches as compared to a target human mTOR.

The method above, wherein the mTOR-specific antisense oligonucleotides reduce a mTOR transcript level by at least 60%, or at least 70%, or at least 80%, or at least 90%.

The method above, wherein the mTOR-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

The method above, wherein the antisense oligonucleotide is conjugated to a polyethylene glycol, a lipid, or a triantennary N-acetyl-galactosamine.

The method above, comprising antisense oligonucleotides in a carrier of sterile water for injection, saline, isotonic saline, or a combination thereof, and wherein the antisense oligonucleotide is stable for at least 14 days in carrier at 37° C.

The method above, comprising antisense oligonucleotides substantially free of excipients.

The method above, wherein the agent for inhibiting mTOR is rapamycin, everolimus, temsirolimus, sirolimus, deforolimus, ridaforolimus, zotarolimus, torkinib, samotolisib, omipalisib, apitolisib, vistusertib, dactolisib, gedatolisib, voxtalisib, chrysophanic, or a combination thereof.

The method above, wherein the agent for inhibiting mTOR is 1-[4-[4-(1-Oxopropyl)-1-piperazinyl]-3-(trifluoromethyl)phenyl]-9-(3-quinolinyl)-benzo[h]-1,6-naphthyridin-2(1H)-one, 3-[4-(4-Morpholinylpyrido[3′,2′:4,5]furo[3,2-d]pyrimidin-2-yl]phenol hydrochloride, N-[4-[4-(4-Morpholinyl)-1-[1-(3-pyridinylmethyl)-4-piperidinyl]-1H-pyrazolo[3,4-d]pyrimidin-6-yl]phenyl]-carbamic acid methyl ester dihydrochloride, 2,4-Difluoro-N-[2-methoxy-5-[4-(4-pyridazinyl)-6-quinolinyl]-3-pyridinyl]benzenesulfonamide (omipalisib), 5-Chloro-N-(2-chloro-4-nitrophenyl)-2-hydroxybenzamide (niclosamide), or a combination thereof.

The method above, wherein the administration is combined with a standard of care treatment for the cancer, wherein the standard of care treatment comprises chemotherapy or radiation therapy, and wherein the administration is performed in the absence of immune checkpoint inhibitors.

The method above, wherein the mTOR-specific antisense oligonucleotide and agent for inhibiting mTOR are each administered concurrently, simultaneously, sequentially, or separately in time.

The method above, wherein the administration decreases mortality rate at month 6, 12, 18, 24, 30, or 36.

The method above, wherein the administration increases survival rate at month 6, 12, 18, 24, 30, or 36.

### BRIEF DESCRIPTION OF THE DRAWINGS

FIG. 1 shows highly significant improvement in overall survival for sarcoma patients with reduced mTOR.

FIG. 2 shows highly significant improvement in overall survival for sarcoma patients in the quartile with lowest mTOR.

FIG. 3 shows highly significant improvement in overall survival for sarcoma patients with below median mTOR.

FIG. 4 shows highly significant improvement in overall survival for lung squamous cell carcinoma patients with reduced mTOR when CD4+ memory T-cells were enriched above median, as measured in a tumor microenvironment.

FIG. 5 shows highly significant improvement in overall survival for lung squamous cell carcinoma patients for which eosinophils were reduced below median, as measured in a tumor microenvironment.

FIG. 6 shows highly significant improvement in overall survival for pancreatic ductal adenocarcinoma patients with reduced mTOR when CD8+ T-cells were enriched above median, as measured in a tumor microenvironment.

FIG. 7 shows highly significant improvement in overall survival for pancreatic ductal adenocarcinoma patients for which tumor mutation burden was reduced below median.

FIG. 8 shows highly significant improvement in overall survival for pancreatic ductal adenocarcinoma patients for which neoantigen load was increased above median.

FIG. 9 shows highly significant improvement in overall survival for sarcoma patients for which tumor mutation burden was reduced below median.

FIG. 10 shows highly significant improvement in overall survival for sarcoma patients for which neoantigen load was reduced below median.

FIG. 11 shows highly significant improvement in overall survival for sarcoma patients for which mesenchymal stem cells were increased above median.

FIG. 12 shows highly significant improvement in overall survival for sarcoma patients for which Type 1 T-helper cells were increased above median.

FIG. 13 shows highly significant improvement in overall survival for sarcoma patients for which Type 1 T-helper cells and mesenchymal stem cells were increased above median.

FIG. 14 shows highly significant improvement in overall survival for sarcoma patients for which Type 1 T-helper cells and mesenchymal stem cells were increased above median, while tumor mutation burden was reduced below median.

FIG. 15 shows highly significant improvement in overall survival for sarcoma patients for which Type 1 T-helper cells were increased above median, while neoantigen load was reduced below median.

### DETAILED DESCRIPTION OF THE DISCLOSURE

This invention relates to methods, agents and uses of mTOR therapies for use against cancer.

This invention provides methods, agents and uses for treating cancer. Embodiments of this invention disclose methods, agents and uses for inhibiting or suppressing mTOR, which can provide improved clinical outcomes for cancer. This invention provides stable formulations of new mTOR agents, including antisense oligonucleotide compositions, as well as combinations with other therapies for treating cancer. This invention includes biomarker methods for selecting subjects who would benefit from use of mTOR inhibitor agents and therapies.

This invention includes new agents and methods for inhibiting mTOR. The novel structures and compositions of this disclosure for inhibiting mTOR can provide novel therapeutic methods. Novel structures of this invention for inhibiting mTOR include antisense oligonucleotides and chemically-modified structures thereof.

In some embodiments, combination of the novel antisense agents with known mTOR inhibitors can supply new therapies.

This invention includes methods to select subjects who would benefit from use of mTOR inhibitor agents and mTOR therapies. A range of specific biomarkers can be used to identify those who would benefit from mTOR therapy. In certain embodiments, specific biomarkers and combinations of biomarkers can be used to select patients or sub-populations who benefit from the mTOR therapy.

In some aspects, therapies disclosed herein can be specific to sarcoma as determined by association with tumor microenvironment components Th1, MSC, and mutational burden. For example, therapies include methods, agents and uses of mTOR inhibitors for sarcoma, specifically sarcoma having low mutational burden, Type 1 T-helper (Th1) enriched sarcoma, and mesenchymal stem cell (MSC) enriched sarcoma.

Components of a tumor microenvironment can be determined by means known in the art, including analysis of fresh frozen and FFPE tissue samples by various techniques, microarray screening, immune cell and subtype analysis by various techniques, as well as genome stability, TMB, expression profiling, and various NGS techniques.

In further aspects, this disclosure provides novel therapeutic agents as mTOR inhibitors including mTOR-specific antisense oligonucleotides.

In some embodiments, this disclosure provides mTOR agents which can be used in various compositions including injection formulations and nanoparticle formulations. The mTOR agents of this disclosure may be used by various routes including intravenous, subcutaneous, intramuscular, intrathecal, and intracranial. In certain embodiments, agents may be encapsulated in a nanoparticle, wherein the size of the nanoparticle is less than about 100 nm, or less than about 80 nm, or less than about 50 nm.

In some embodiments, mTOR agents of this disclosure can be used by intracranial, intrathecal or intraventricular continuous infusion.

Embodiments of this invention include agents for suppressing or inhibiting mTOR, including mTOR-specific antisense oligonucleotides.

This invention includes methods for treating or ameliorating a symptom of cancer in a human or animal subject in need by administering a therapeutically sufficient amount of a pharmaceutical composition comprising an agent for suppressing or inhibiting mTOR to the subject.

Additional embodiments provide agents for suppressing or inhibiting mTOR for treating or ameliorating a symptom of cancer in a human or animal subject in need.

In certain embodiments, this invention provides uses of a composition comprising an agent for suppressing or inhibiting mTOR in the preparation of a medicament for treating or ameliorating a symptom of cancer in a human or animal subject in need.

Therapies of this invention can have impact on various cancers, including sarcoma, lung cancer, ovarian cancer, pancreatic cancer, and similar pathologies.

Further aspects of this disclosure include methods, agents and uses which employ one or more biomarkers to select subjects who benefit from a therapy or use of agents.

In some embodiments, biomarkers can include a level of a tumor mutation burden (TMB), a level of a tumor neoantigen, a level of a tumor-associated immune cell, and combinations thereof.

Certain embodiments directly involve biomarkers including a level of a basophil cell, a B-cell, a T-cell, a T-helper cell (Th), an eosinophil cell, a macrophage cell, a mesenchymal stem cell, and/or a combination thereof.

Further embodiments directly utilize biomarkers such as a level of a CD4+ cell, a memory T-cell, a CD8+ cell, a natural killer T-cell, a regulatory T-cell, a type 1 T-helper cell (Th1), a type 2 T-helper cell (Th2), or a combination thereof, wherein the levels can be determined in a tumor environment, or microenvironment.

Embodiments of this invention further contemplate novel therapies and agents against sarcoma by selecting patients using biomarkers such as tumor mutation burden (TMB), tumor neoantigen level, mesenchymal stem cell, type 1 T-helper cell (Th1), or a combination thereof, wherein the biomarkers can be determined in a tumor environment, or tumor microenvironment.

In some embodiments, methods, agents and uses of this invention can provide potency against lung squamous cell carcinoma, where biomarkers such as a level of a CD4+ cell, a level of a memory T-cell, a level of an eosinophil cell, or a combination thereof, can be determined to select a sub-population who benefits from the therapy.

In further embodiments, this disclosure provides methods, agents and uses for pancreatic cancer, where biomarkers such as a level of a CD8+ cell, a tumor mutation burden (TMB), or a combination thereof are determined to guide and deliver benefits of the therapy to selected patients.

In additional embodiments, some methods, agents and uses of this invention work against ovarian cancer, where biomarkers such as a level of a natural killer T-cell, a tumor neoantigen, or a combination thereof can be determined to select subjects benefitting from the methods, agents and uses.

Embodiments of this invention further encompass agents for suppressing or inhibiting mTOR including mTOR-specific antisense oligonucleotides. mTOR-specific antisense oligonucleotides can be chemically-modified, including LNA variants, and gapmer variants. Methods and formulations can include pooling of mTOR antisense oligonucleotides, as well as combining antisense oligonucleotides with other agents for inhibiting mTOR. In certain embodiments, antisense oligonucleotides can be used in combination with other agents for inhibiting mTOR, where each agent can be administered concurrently, simultaneously, sequentially, or separately in time.

Formulations of this disclosure include supplying one or more agents for suppressing or inhibiting mTOR in a carrier of sterile water for injection, saline, isotonic saline, or a combination thereof. In certain embodiments, a formulation can be substantially free of excipients. Formulations of this disclosure can also be stable for at least 14 days in such carriers at 37° C.

Embodiments of this invention further encompass use or administration of compositions or formulations of agents for suppressing or inhibiting mTOR in combination with a standard of care treatment for a cancer, wherein the standard of care treatment can include chemotherapy or radiation therapy.

Embodiments of this invention further encompass use or administration of compositions or formulations of agents for suppressing and/or inhibiting mTOR which can decrease selected patient mortality rates at month 6, 12, 18, 24, 30, or 36.

Embodiments of this invention further encompass use or administration of compositions or formulations of agents for suppressing and/or inhibiting mTOR which can increase selected patient survival rate at month 6, 12, 18, 24, 30, or 36.

This disclosure provides compositions or formulations of agents for suppressing and/or inhibiting mTOR for use as a single agent, where subjects may also be selected with biomarkers.

A composition or formulation of this invention may be prepared and/or used in the absence of any immune checkpoint inhibitor. Therapies of this invention can be performed in the absence of any immune checkpoint inhibitor.

Embodiments of this invention include compositions of mTOR antisense oligonucleotides for use in the absence of any immune checkpoint inhibitor drug.

### Additional Therapies for Cancer

Additional therapies for cancer of this disclosure include methods for treating or ameliorating a symptom of cancer in a human or animal subject in need by selecting a subject who benefits from the method based on one or more biomarkers, and administering a therapeutically sufficient amount of a pharmaceutical composition comprising a combination based on agents for suppressing mTOR and agents for inhibiting mTOR.

Embodiments provide combinations based on agents for suppressing mTOR and agents for inhibiting mTOR for treating or ameliorating a symptom of cancer in a human or animal subject in need, wherein the subjects who benefit from the method can be selected based on one or more biomarkers.

In certain embodiments, agents for suppressing mTOR and agents for inhibiting mTOR can be used in combination, where each agent can be administered concurrently, simultaneously, sequentially, or separately in time.

In certain embodiments, this disclosure includes compositions comprising a combination of agents for suppressing mTOR and an agent for inhibiting mTOR in the preparation of a medicament for treating or ameliorating a symptom of cancer in a human or animal subject in need, wherein the subject is selected who benefits from the method based on one or more biomarkers.

The method, agent or use above, wherein the cancer is a sarcoma, a lung cancer, an ovarian cancer, or a pancreatic cancer.

Biomarkers which can be determined in such embodiments include a level of a tumor mutation burden (TMB), a level of a tumor neoantigen, a level of a tumor-associated immune cell, or a combination thereof. In some embodiments, biomarkers which can be determined are a level of a basophil cell, a B-cell, a T-cell, a T-helper cell (Th), an eosinophil cell, a macrophage cell, a mesenchymal stem cell, or a combination thereof. In certain embodiments, biomarkers which may be used include a level of a CD4+ cell, a memory T-cell, a CD8+ cell, a natural killer T-cell, a regulatory T-cell, a type 1 T-helper cell (Th1), a type 2 T-helper cell (Th2), or a combination thereof.

For therapies using formulations against sarcoma, biomarkers which may be determined include a level of a tumor mutation burden (TMB), a tumor neoantigen level, a mesenchymal stem cell, a type 1 T-helper cell (Th1), or a combination thereof.

For therapies using formulations against lung squamous cell carcinoma, biomarkers which may be determined include a level of a CD4+ cell, a memory T-cell, an eosinophil cell, or a combination thereof.

For therapies using formulations against pancreatic cancer, biomarkers which may be determined include a level of a CD8+ cell, a tumor mutation burden (TMB), or a combination thereof.

For therapies using formulations against ovarian cancer, biomarkers which may be determined include a level of a natural killer T-cell, a tumor neoantigen, or a combination thereof.

Examples of agents for inhibiting mTOR include rapamycin, everolimus, temsirolimus, sirolimus, deforolimus, ridaforolimus, zotarolimus, torkinib, samotolisib, omipalisib, apitolisib, vistusertib, dactolisib, gedatolisib, voxtalisib, chrysophanic, and combinations thereof.

Examples of agents for inhibiting mTOR include 1-[4-[4-(1-Oxopropyl)-1-piperazinyl]-3-(trifluoromethyl)phenyl]-9-(3-quinolinyl)-benzo[h]-1,6-naphthyridin-2(1H)-one, 3-[4-(4-Morpholinylpyrido[3′,2′:4,5]furo[3,2-d]pyrimidin-2-yl]phenol hydrochloride, N-[4-[4-(4-Morpholinyl)-1-[1-(3-pyridinylmethyl)-4-piperidinyl]-1H-pyrazolo[3,4-d]pyrimidin-6-yl]phenyl]-carbamic acid methyl ester dihydrochloride, 2,4-Difluoro-N-[2-methoxy-5-[4-(4-pyridazinyl)-6-quinolinyl]-3-pyridinyl]benzenesulfonamide (omipalisib), 5-Chloro-N-(2-chloro-4-nitrophenyl)-2-hydroxybenzamide (niclosamide), or a combination thereof.

Therapies for cancer of this invention can be combined with a standard of care treatment for the same cancer. Examples of standard-of-care treatment include chemotherapy and radiation therapy.

Embodiments of this invention which encompass use or administration of compositions or formulations of agents for suppressing and/or inhibiting mTOR may decrease selected patient mortality rates at month 6, 12, 18, 24, 30, or 36.

Embodiments of this invention which encompass use or administration of compositions or formulations of agents for suppressing and/or inhibiting mTOR may increase selected patient survival rate at month 6, 12, 18, 24, 30, or 36.

This disclosure includes methods for treating or ameliorating a symptom of cancer in a human or animal subject in need, the methods comprising selecting a subject who benefits from the method based on one or more biomarkers, and administering a therapeutically sufficient amount of a pharmaceutical composition comprising an agent for suppressing mTOR in the subject, in the absence of any immune checkpoint inhibitor.

A composition or formulation of this invention may be prepared and/or used in the absence of any immune checkpoint inhibitor. Therapies of this invention can be performed in the absence of any immune checkpoint inhibitor.

Embodiments of this invention include compositions of mTOR antisense oligonucleotides for use in the absence of any immune checkpoint inhibitor drug.

This invention includes uses of a composition comprising an agent for suppressing mTOR in the preparation of a medicament for treating or ameliorating a symptom of cancer in a human or animal subject in need, wherein the subject who benefits from the method may be selected based on one or more biomarkers.

mTOR-Specific Antisense Oligonucleotides

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone.

Human mTOR-specific phosphorothioate antisense oligodeoxynucleotides can be used to suppress or inhibit mTOR.

For example, reference sequences for mTOR are given at NG_033239.1 and NM_004958.4.

Antisense oligodeoxynucleotides are short strings of DNA that can be designed to downregulate gene expression by interfering with the translation of a specific encoded protein at the mRNA level. In some embodiments, all 3′-5′ linkages may be modified to phosphorothioates.

In some embodiments, an agent for suppressing mTOR may be an mTOR-specific antisense oligonucleotide complementary to a mTOR transcript and 15-30 nucleotides in length.

In certain embodiments, an agent for suppressing mTOR may be an mTOR-specific antisense oligonucleotide complementary to a mTOR pre-RNA, pre-mRNA or mRNA and 18-21 nucleotides in length.

Examples of agents of this disclosure for suppressing or inhibiting mTOR include mTOR-specific antisense oligonucleotides given in SEQ ID NOs:1-20 in Table 1, and chemically-modified variants thereof, LNA variants thereof, gapmer variants thereof, and pooling and combinations thereof.

TABLE 1
mTOR-specific antisense oligonucleotides

| SEQ ID | | ANTISENSE SEQUENCE |
|---|---|---|
| NO: | Ref. | 5′→3” |
| 1 | 719-738 | TTCGGCTCACGCTGGGTTGT |
| 2 | 974-993 | CGAGGTTTTGTTCCGAAGCC |
| 3 | 985-1004 | GGGTAATGTGACGAGGTTTT |
| 4 | 986-1005 | GGGGTAATGTGACGAGGTTT |
| 5 | 1163-1182 | CACACCTGATCAAATTTCTC |
| 6 | 1164-1183 | GCACACCTGATCAAATTTCT |
| 7 | 2670-2689 | CTGCTCAGTCTTCAGAAAAT |
| 8 | 2671-2690 | TCTGCTCAGTCTTCAGAAAA |
| 9 | 4121-4140 | AAGTTTAAGAGGGTCTGTGT |
| 10 | 5115-5134 | TGTTGGCAGAGGATGGTCAA |
| 11 | 5117-5136 | ACTGTTGGCAGAGGATGGTC |
| 12 | 5355-5374 | TTTGGGGATTGTGCTCTCAT |
| 13 | 5467-5486 | GGTTCTGATGTTTGTAGTGT |
| 14 | 5468-5487 | TGGTTCTGATGTTTGTAGTG |
| 15 | 5473-5492 | GGGCTTGGTTCTGATGTTTG |
| 16 | 5474-5493 | CGGGCTTGGTTCTGATGTTT |
| 17 | 6481-6500 | TGGGGTCATATGTTCCTGGC |
| 18 | 6482-6501 | TTGGGGTCATATGTTCCTGG |
| 19 | 7769-7788 | GGTTTCAGTTTAGTGGAAGC |
| 20 | 8297-8316 | TGAATTGAAGCGTGTGAGTC |

The sequences of Table 1 can be chemically-modified to provide active variants thereof, LNA variants thereof, as well as gapmer variants thereof, as known in the art. The sequences of Table 1 can be used in any combination as active agents, such as pooling combinations.

For example, a synthetic mTOR-specific antisense oligonucleotide of this disclosure may have one or all 3′-5′ linkages modified to phosphorothioate.

It is understood that additional antisense oligonucleotides can be constructed based on the mTOR gene sequence. In some embodiments, certain criteria for selection of mTOR target sites can be used as follows:

$$40 \% <= GC , \% <= 60 \% ;$$

No GGGG in the target sequence;
Average unpaired probability for target site nucleotides >=0.5;
For each peak in the accessibility profile above the threshold probability of 0.5, all sites targeted to this same peak can be ranked by their average unpaired probability, and at most n sites may be selected for each peak, where n is determined by max([width of peak/site length], 2).

In some embodiments, a mTOR-specific antisense oligonucleotide of this invention may have no more than one or two mismatches as compared to a target human mTOR.

In certain embodiments, a mTOR-specific antisense oligonucleotide of this invention may reduce a mTOR transcript level by at least 60%, or at least 70%, or at least 80%, or at least 90%.

In some aspects, an mTOR-specific antisense oligonucleotides may have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

In certain embodiments, an antisense oligonucleotide may be conjugated to a polyethylene glycol, a lipid, or a triantennary N-acetyl-galactosamine.

### Methods and Compositions for Cancer

The agents of this invention can be used for treating cancer or ameliorating the symptoms of cancer in a human subject or animal in need. The agents may be prepared in a pharmaceutical composition for infusion.

A pharmaceutical composition for infusion may include an agent for inhibiting or suppressing mTOR, and be administered in a therapeutically sufficient amount to the subject.

Examples of mTOR inhibitors include antisense oligonucleotides and pharmaceutically acceptable salts forms, esters, and variants thereof.

Pharmaceutical agents or active substances of this disclosure may be dissolved or suspended in a physiological solvent or in any other appropriate solvent. The agents may in the form of a free base, or a salt, hydrate, ester, amide, enantiomer, isomer, tautomer, polymorph, prodrug, or derivative of these compounds. The above mentioned agents as well as combinations thereof can be used in the apparatuses, methods, kits, combinations, and compositions herein described.

Compositions of this invention can contain compounds to be infused to the subject formulated as an injectable formulation, for example, an aqueous solution or suspension of the compounds suitable for intravenous delivery. When preparing the composition for injection, particularly for intravenous delivery, illustratively, the continuous phase comprises an aqueous solution of tonicity modifiers, buffered to a pH below 7, for example, or below 6, for example. The tonicity modifiers comprise, for example, sodium chloride, glucose, mannitol, trehalose, glycerol, or other pharmaceutical agents that renders the osmotic pressure of the formulation isotonic with blood.

The system of this invention can contain a preservative added to the formulation. A preservative includes benzalkonium chloride, propylparaben, butylparaben. chlorobutanol, belizyl alcohol, phenol. sodium benzoate, or EDTA.

The composition of this disclosure can contain a pharmaceutically acceptable carrier. The carrier materials that can be employed in making the compositions of the present invention are any of those commonly used excipients in pharmaceutics and should be selected on the basis of compatibility with the pharmaceutical agent and the release pro-file properties of the desired dosage form.

A composition of this invention can contain excipients such as are given in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N.Y., 1980.

### Formulation of Antisense

An antisense oligonucleotide may be supplied as a lyophilized powder in 50 mL glass vials in different quantities. Each vial can be identified by the name of the investigational product, trial number, dosing group, mode of application, quantity of ASO contained (in mg), total volume after dissolving (in mL) and resulting concentration (in M), name of sponsor, name of manufacturer, batch number, vial number, storage temperature, and expiry date. Medication can be provided in closed units, packaged separately for each concentration. Packages may contain the appropriate vial(s) and all necessary components of the application system (i.e., infusion system). ASO lyophilized powder can be dissolved in isotonic (0.9%) aqueous sodium chloride prior to use.

In some examples and embodiments, an agent may be an mTOR-specific antisense oligonucleotide selected from SEQ ID NOs:1-20 and chemically-modified variants thereof, and administered or used by infusion at a dose of 4 μl/min at a dose level of 10 μM on Days 1 to 7, or at a dose of 20 μM on Days 1 to 7, or at a dose of 40 μM on Days 1 to 7, or at a dose of 80 μM on Days 1 to 7.

In some examples and embodiments, an agent may be an mTOR-specific antisense oligonucleotide selected from SEQ ID NOs:1-20 and chemically-modified variants thereof, and administered or used by infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

In some embodiments, an agent may be an mTOR-specific antisense oligonucleotide selected from SEQ ID NOs:1-20, and chemically-modified variants thereof, and administered by injection at concentrations of 61.43 mg/ml (10 PM), 1 mg/ml, 7.35 mg/ml, 15 mg/ml, or 18.23 mg/ml.

In further embodiments, an agent may be an mTOR-specific antisense oligonucleotide selected from SEQ ID NOs:1-20 and chemically-modified variants thereof, and administered or used by infusion, either singly, in combination with a formulation-compatible drug, or in combination with standard of care therapies.

An agent of this disclosure may be a pharmaceutically-acceptable salt, salt polymorph, ester, or isomer, and one or more pharmaceutically acceptable excipients. Excipients may comprise any one or more pharmaceutically acceptable excipients selected from diluents, stabilizers, disintegrants and anticaking agents. In some embodiments, the excipients may comprise any one or more of microcrystalline cellulose, polysorbate 80, crospovidone, croscarmellose sodium, and magnesium stearate.

A pharmaceutical compositions may contain an mTOR inhibitor as well as a carrier. The carrier may be sterile water for injection, saline, isotonic saline, or a combination thereof.

Importantly, a composition of this disclosure may be substantially free of excipients. Compositions of this invention which are substantially free of excipients can be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

Embodiments of this invention further contemplate therapeutic modalities in which a composition of this invention is administered or utilized in combination with a standard of care therapy for the disease.

In further embodiments, a therapeutically effective amount of an antisense agent for inhibiting or suppressing expression of mTOR can be from 0.1 to 3000 mg per day, or 1 to 1000 mg per day, or 2 to 500 mg per day, or 2 to 200 mg per day.

In certain embodiments, a formulation of an antisense agent for inhibiting or suppressing expression of mTOR can have a concentration of from 0.05 to 50 PM, or 0.1 to 25 μM, or 0.1 to 10 μM, or 0.1 to 7.5 μM, or 0.1 to 5 μM.

In certain embodiments, a method for using an antisense agent for inhibiting or suppressing expression of mTOR can use an effective dosage amount of from 1 to 1000 mg/m²/day, or from 1 to 500 mg/m²/day, or from 1 to 250 mg/m²/day, or from 1 to 100 mg/m²/day, or from 1 to 50 mg/m²/day. Mean human body surface area can be about 1.6 to 1.9 m².

In additional embodiments, a method for using an antisense agent for inhibiting or suppressing expression of mTOR can use an effective dosage amount of from 0.05 to 40 mg/kg/day, or from 0.1 to 30 mg/kg/day, or from 0.2 to 20 mg/m²/day, or from 0.3 to 10 mg/m²/day, or from 0.5 to 5 mg/m²/day. Mean human body weight can be about 60 kg.

In certain embodiments, agents of this disclosure for inhibiting or suppressing expression of mTOR may be prepared from a lyophilized powder of the agent.

In some examples and embodiments, an agent may be a mTOR-specific antisense oligonucleotide selected from SEQ ID NOs:1-20 and chemically-modified variants thereof, and administered or used by injection or infusion at a dose of 4 μl/min at a dose level of 10 μM on the Days 1 to 7, or at a dose of 20 μM on Days 1 to 7, or at a dose of 40 μM on Days 1 to 7, or at a dose of 80 μM on Days 1 to 7.

In certain embodiments, a mTOR-specific antisense oligonucleotide selected from SEQ ID NOs:1-20 and chemically-modified variants thereof may be supplied as a sterile lyophilizate for solution prior to administration in 20R glass vials with a quantity of 250 mg/vial. The lyophilizate may be reconstituted aseptically in sterile, preservative-free isotonic NaCl solution. Antisense oligonucleotide solution can be administered every 14 days using a portable pump system as a continuous i.v. infusion on days 4-7 according to a 4-days-on, 10-days-off schedule. A schedule may be 7 d on/7d off and 4 d on/10 d off scheduling. A dose may be 40, 80, 160, 140, 190, 250, 330 mg.

In certain embodiments, an agent may be a mTOR gene sequence-specific antisense oligonucleotide selected from SEQ ID NOs:1-20 and chemically-modified variants thereof, and administered or used by injection or infusion at a dose of 40, 80, 160, 140, 190, 250, 330 mg/m² on Days 1 to 7, or at a dose of 40, 80, 160, 140, 190, 250, 330 mg/m² on Days 1 to 4.

In some examples and embodiments, an agent may be a mTOR gene sequence-specific antisense oligonucleotide selected from SEQ ID NOs:1-20 and chemically-modified variants thereof, and administered or used by injection or infusion at a dose of 4 μl/min, or 2-8 μl/min, at a dose level of 2 μM on Days 1 to 7, or at a dose of 4 μM on Days 1 to 7, or at a dose of 8 μM on Days 1 to 7, or at a dose of 10 μM on Days 1 to 7.

As used herein, the term chemically-modified variants can refer to LNA variants and gapmer variants. Antisense agents of this disclosure can be used by pooling in a formulation, or used in any combination.

Numbered embodiments of this invention may include:

1) A method for treating or ameliorating a symptom of cancer in a human or animal subject in need, the method comprising: administering a therapeutically sufficient amount of a pharmaceutical composition comprising an agent for suppressing mTOR to the subject.

2) An agent for suppressing mTOR for treating or ameliorating a symptom of cancer in a human or animal subject in need.

3) Use of a composition comprising an agent for suppressing mTOR in the preparation of a medicament for treating or ameliorating a symptom of cancer in a human or animal subject in need.

4) The method, agent or use of any of embodiments 1-3, wherein the cancer is a sarcoma, a lung cancer, an ovarian cancer, or a pancreatic cancer.

5) The method, agent or use of any of embodiments 1-4, comprising using one or more biomarkers to select subjects who benefit from the method, agent or use.

6) The method, agent or use of any of embodiments 1-5, wherein the one or more biomarkers are a level of a tumor mutation burden (TMB), a tumor neoantigen, a tumor-associated immune cell, or a combination thereof.

7) The method, agent or use of any of embodiments 1-6, wherein the one or more biomarkers are a level of a basophil cell, a B-cell, a T-cell, a T-helper cell (Th), an eosinophil cell, a macrophage cell, a mesenchymal stem cell, or a combination thereof, as determined in a tumor microenvironment.

8) The method, agent or use of any of embodiments 1-7, wherein the one or more biomarkers are a level of a CD4+ cell, a memory T-cell, a CD8+ cell, a natural killer T-cell, a regulatory T-cell, a type 1 T-helper cell (Th1), a type 2 T-helper cell (Th2), or a combination thereof, as determined in a tumor microenvironment.

9) The method, agent or use of any of embodiments 1-8, wherein the cancer is a sarcoma and the one or more biomarkers are a level of a tumor mutation burden (TMB), a tumor neoantigen level, a mesenchymal stem cell determined in a tumor microenvironment, a type 1 T-helper cell (Th) determined in a tumor microenvironment, or a combination thereof.

10) The method, agent or use of any of embodiments 1-9, wherein the cancer is a lung squamous cell carcinoma and the one or more biomarkers are a level of a CD4+ cell determined in a tumor microenvironment, a memory T-cell determined in a tumor microenvironment, an eosinophil cell determined in a tumor microenvironment, or a combination thereof.

11) The method, agent or use of any of embodiments 1-10, wherein the cancer is a pancreatic cancer and the one or more biomarkers are a level of a CD8+ cell determined in a tumor microenvironment, a tumor mutation burden (TMB), or a combination thereof.

12) The method, agent or use of any of embodiments 1-11, wherein the cancer is an ovarian cancer and the one or more biomarkers are a level of a natural killer T-cell determined in a tumor microenvironment, a tumor neoantigen determined in a tumor microenvironment, or a combination thereof.

13) The method, agent or use of any of embodiments 1-12, wherein the agent for suppressing mTOR is an mTOR-specific antisense oligonucleotide complementary to a mTOR transcript and 15-30 nucleotides in length, or complementary to a mTOR pre-RNA, pre-mRNA or mRNA and 18-21 nucleotides in length.

14) The method, agent or use of any of embodiments 1-13, wherein the agent for suppressing mTOR is an mTOR-specific antisense oligonucleotide comprising any of the 20 nucleotide length sequences in Table 1 and chemically-modified variants thereof, LNA variants thereof, gapmer variants thereof, and pooling and combinations thereof.

15) The agent, use or method of any of embodiments 1-14, wherein the mTOR-specific antisense oligonucleotides have no more than one or two mismatches as compared to a target human mTOR.

16) The agent, use or method of any of embodiments 1-15, wherein the mTOR-specific antisense oligonucleotides reduce a mTOR transcript level by at least 60%, or at least 70%, or at least 80%, or at least 90%.

17) The agent, use or method of any of embodiments 1-16, wherein the mTOR-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

18) The agent, use or method of any of embodiments 1-17, wherein the antisense oligonucleotide is conjugated to a polyethylene glycol, a lipid, or a triantennary N-acetyl-galactosamine.

19) The method, agent or use of any of embodiments 1-18, comprising antisense oligonucleotide in a carrier of sterile water for injection, saline, isotonic saline, or a combination thereof.

20) The method, agent or use of any of embodiments 1-19, wherein the antisense oligonucleotides are substantially free of excipients.

21) The method, agent or use of any of embodiments 1-20, wherein the antisense oligonucleotides are stable for at least 14 days in carrier at 37° C.

22) The method, agent or use of any of embodiments 1-21, wherein the administration or use of the composition is combined with a standard of care treatment for the cancer, wherein the standard of care treatment comprises chemotherapy or radiation therapy, and wherein the administration or use of the composition is performed in the absence of immune checkpoint inhibitors.

23) The method, agent or use of any of embodiments 1-22, wherein the administration or use decreases mortality rate at month 6, 12, 18, 24, 30, or 36.

24) The method, agent or use of any of embodiments 1-23, wherein the administration or use increases survival rate at month 6, 12, 18, 24, 30, or 36.

25) A method for treating or ameliorating a symptom of cancer in a human or animal subject in need, the method comprising: administering a therapeutically sufficient amount of a pharmaceutical composition comprising an mTOR-specific antisense oligonucleotide in combination with an agent for inhibiting mTOR to the subject.

26) The method of embodiment 25, wherein the cancer is a sarcoma, a lung cancer, an ovarian cancer, or a pancreatic cancer.

27) The method of any of embodiments 25-26, comprising using one or more biomarkers to select subjects who benefit from the method.

28) The method of any of embodiments 25-27, wherein the one or more biomarkers are a level of a tumor mutation burden (TMB), a tumor neoantigen, a tumor-associated immune cell, or a combination thereof.

29) The method of any of embodiments 25-28, wherein the one or more biomarkers are a level of a basophil cell determined in a tumor microenvironment, a B-cell determined in a tumor microenvironment, a T-cell determined in a tumor microenvironment, a T-helper cell (Th) determined in a tumor microenvironment, an eosinophil cell determined in a tumor microenvironment, a macrophage cell determined in a tumor microenvironment, a mesenchymal stem cell determined in a tumor microenvironment, or a combination thereof.

30) The method of any of embodiments 25-29, wherein the one or more biomarkers are a level of a CD4+ cell determined in a tumor microenvironment, a memory T-cell determined in a tumor microenvironment, a CD8+ cell determined in a tumor microenvironment, a natural killer T-cell determined in a tumor microenvironment, a regulatory T-cell determined in a tumor microenvironment, a type 1 T-helper cell (Th1) determined in a tumor microenvironment, a type 2 T-helper cell (Th2) determined in a tumor microenvironment, or a combination thereof.

31) The method of any of embodiments 25-30, wherein the cancer is a sarcoma and the one or more biomarkers are a level of a tumor mutation burden (TMB), a tumor neoantigen level, a mesenchymal stem cell determined in a tumor microenvironment, a type 1 T-helper cell (Th) determined in a tumor microenvironment, or a combination thereof.

32) The method of any of embodiments 25-31, wherein the cancer is a lung squamous cell carcinoma and the one or more biomarkers are a level of a CD4+ cell determined in a tumor microenvironment, a memory T-cell determined in a tumor microenvironment, an eosinophil cell determined in a tumor microenvironment, or a combination thereof.

33) The method of any of embodiments 25-32, wherein the cancer is a pancreatic cancer and the one or more biomarkers are a level of a CD8+ cell determined in a tumor microenvironment, a tumor mutation burden (TMB), or a combination thereof.

34) The method of any of embodiments 25-33, wherein the cancer is an ovarian cancer and the one or more biomarkers are a level of a natural killer T-cell determined in a tumor microenvironment, a tumor neoantigen determined in a tumor microenvironment, or a combination thereof.

35) The method of any of embodiments 25-34, wherein the mTOR-specific antisense oligonucleotide is complementary to a mTOR transcript and 15-30 nucleotides in length, or complementary to a mTOR pre-RNA, pre-mRNA or mRNA and 18-21 nucleotides in length.

36) The method of any of embodiments 25-35, wherein the mTOR-specific antisense oligonucleotide comprises any of the 20 nucleotide length sequences in Table 1 and chemically-modified variants thereof, LNA variants thereof, gapmer variants thereof, and pooling and combinations thereof.

37) The method of any of embodiments 25-36, wherein the mTOR-specific antisense oligonucleotides have no more than one or two mismatches as compared to a target human mTOR.

38) The method of any of embodiments 25-37, wherein the mTOR-specific antisense oligonucleotides reduce a mTOR transcript level by at least 60%, or at least 70%, or at least 80%, or at least 90%.

39) The method of any of embodiments 25-38, wherein the mTOR-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

40) The method of any of embodiments 25-39, wherein the antisense oligonucleotide is conjugated to a polyethylene glycol, a lipid, or a triantennary N-acetyl-galactosamine.

41) The method of any of embodiments 25-40, comprising antisense oligonucleotides in a carrier of sterile water for injection, saline, isotonic saline, or a combination thereof, and wherein the antisense oligonucleotide is stable for at least 14 days in carrier at 37° C.

42) The method of any of embodiments 25-41, comprising antisense oligonucleotides substantially free of excipients.

43) The method of any of embodiments 25-42, wherein the agent for inhibiting mTOR is rapamycin, everolimus, temsirolimus, sirolimus, deforolimus, ridaforolimus, zotarolimus, torkinib, samotolisib, omipalisib, apitolisib, vistusertib, dactolisib, gedatolisib, voxtalisib, chrysophanic, or a combination thereof.

44) The method of any of embodiments 25-43, wherein the agent for inhibiting mTOR is 1-[4-[4-(1-Oxopropyl)-1-piperazinyl]-3-(trifluoromethyl)phenyl]-9-(3-quinolinyl)-benzo[h]-1,6-naphthyridin-2(1H)-one, 3-[4-(4-Morpholinylpyrido[3′,2′:4,5]furo[3,2-d]pyrimidin-2-yl]phenol hydrochloride, N-[4-[4-(4-Morpholinyl)-1-[1-(3-pyridinylmethyl)-4-piperidinyl]-1H-pyrazolo[3,4-d]pyrimidin-6-yl]phenyl]-carbamic acid methyl ester dihydrochloride, 2,4-Difluoro-N-[2-methoxy-5-[4-(4-pyridazinyl)-6-quinolinyl]-3-pyridinyl]benzenesulfonamide (omipalisib), 5-Chloro-N-(2-chloro-4-nitrophenyl)-2-hydroxybenzamide (niclosamide), or a combination thereof.

45) The method of any of embodiments 25-44, wherein the administration is combined with a standard of care treatment for the cancer, wherein the standard of care treatment comprises chemotherapy or radiation therapy, and wherein the administration is performed in the absence of immune checkpoint inhibitors.

46) The method of any of embodiments 25-45, wherein the mTOR-specific antisense oligonucleotide and agent for inhibiting mTOR are each administered concurrently, simultaneously, sequentially, or separately in time.

47) The method of any of embodiments 25-46, wherein the administration decreases mortality rate at month 6, 12, 18, 24, 30, or 36.

48) The method of any of embodiments 25-47, wherein the administration increases survival rate at month 6, 12, 18, 24, 30, or 36.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

It is emphasized herein according to common practice the features of the drawings have arbitrary scale and are intended to cover similar features that may be arbitrarily expanded or reduced.

### EXAMPLES

Example 1. Methods and agents of this disclosure for suppressing mTOR can be used against sarcoma.

A study of clinical results for a total of 259 sarcoma patients having multiple types of sarcoma was performed which showed improvement in overall survival (OS) with reduced mTOR expression. This was a highly surprising result because comparative data from a total of over 7,200 other cancer patients having a wide range of different cancers did not show such improvement with reduced mTOR expression. The patients were treated in the absence of any immune checkpoint inhibitor drugs.

Kaplan-Meier Plotter was used to calculate patient survival to determine the connection between improved overall survival in sarcoma and reduced mTOR expression. Some raw data is available in Kovacs, Transcriptomic datasets of cancer patients treated with immune-checkpoint inhibitors: a systematic review, J. Transl. Med., 2022, May 31, Vol. 20(1), pp. 249.

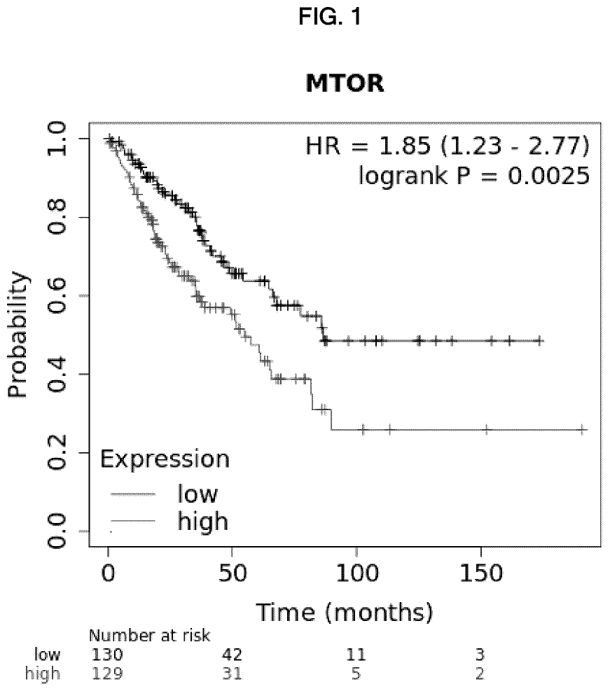

Results of the study for sarcoma are shown in FIG. 1. FIG. 1 shows highly significant (logrank P=0.0025) improvement in overall survival for sarcoma patients with reduced mTOR. The improvement found in overall survival with reduced mTOR expression was a significant increase from 55 months for the high mTOR expression cohort to 87 months for the low mTOR expression cohort. Sarcoma subtypes in this study included all stages, genders, races, grades, mutation burdens, and neoantigen loadings. Expression ranges of the probes were from 345-4010. In this study, no restrictions or exclusions were made for tumor microenvironment immune cell components.

An additional study of clinical results for a total of 3,711 sarcoma patients having multiple types of sarcoma was performed which also showed improvement in overall survival (OS) with reduced mTOR expression. In this study, cBioPortal for Cancer Genomics was used to calculate patient survival to determine the connection between improved overall survival in sarcoma and reduced mTOR expression. Sarcoma types included soft tissue sarcoma (1,987), bone cancer (529), gastrointestinal stromal tumor (395), uterine sarcoma (341), sarcoma (255), endometrial cancer (136), nerve sheath tumor (64), and soft tissue cancer (4). The patients were treated in the absence of any immune checkpoint inhibitor drugs.

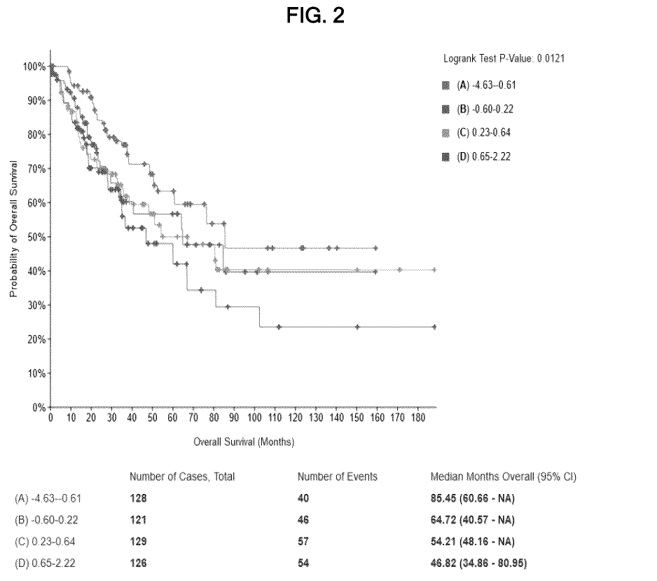

Results of this additional study for sarcoma are shown in FIG. 2. FIG. 2 shows highly significant (logrank P=0.0121) improvement in overall survival for 504 sarcoma patients in the quartile with lowest mTOR. The improvement found in overall survival with reduced mTOR expression was a significant increase from 47 months for the highest mTOR expression quartile D to 85 months for the lowest mTOR expression cohort A.

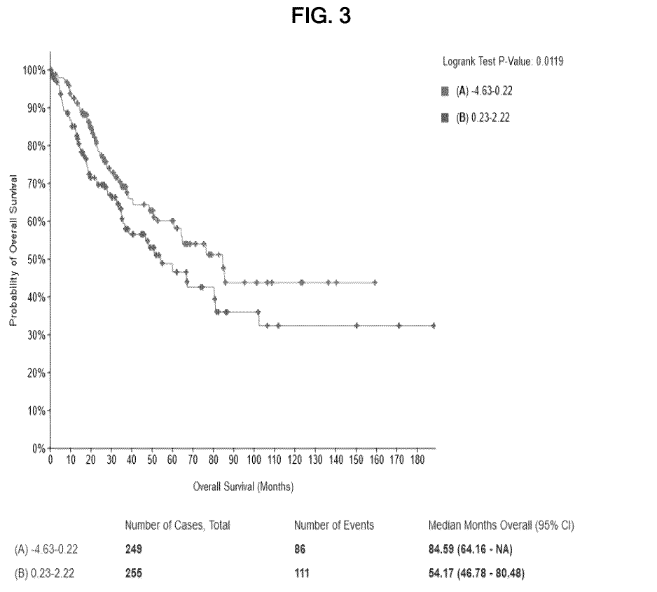

Similar results were found for the same data using median mTOR expression analysis as shown in FIG. 3. FIG. 3 shows highly significant (logrank P=0.0119) improvement in overall survival for 504 sarcoma patients with below median mTOR. The improvement found in overall survival with reduced mTOR expression was a significant increase from 54 months for above median mTOR expression to 85 months for below median mTOR expression. This surprising improvement in overall survival with reduced mTOR was independent of sarcoma type because both the above median and below median mTOR expression groups contained similar numbers and types of sarcoma. This surprising improvement in overall survival with reduced mTOR was also independent of sarcoma anatomical site because both the above median and below median mTOR expression groups contained similar numbers of anatomical locations of sarcoma.

Example 2. Methods and agents of this disclosure for suppressing and inhibiting mTOR can be used against lung squamous cell carcinoma where biomarkers are used to select patients who benefit from the methods and/or agents. Biomarkers of significance against lung squamous cell carcinoma were a level of a CD4+ memory T-cell determined in a tumor microenvironment, and a level of an eosinophil cell determined in a tumor microenvironment.

A study of clinical results for lung squamous cell carcinoma patients was performed which showed improvement in overall survival (OS) with reduced mTOR expression when CD4+ memory T-cells were enriched in a tumor microenvironment. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in lung squamous cell carcinoma and reduced mTOR expression where biomarkers were used to select patients who benefit from the methods and/or agents.

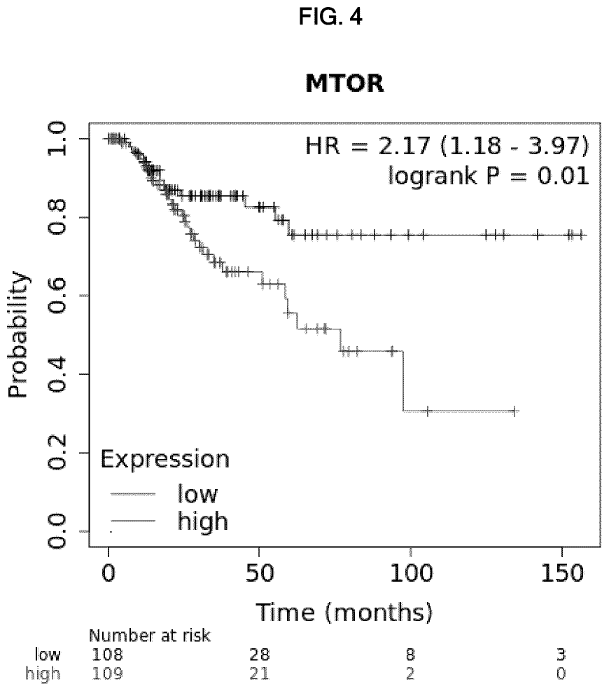

Results of the study for lung squamous cell carcinoma are shown in FIG. 4. FIG. 4 shows highly significant (logrank P=0.0105) improvement in overall survival for patients with reduced mTOR when CD4+ memory T-cells were enriched above median, as measured in a tumor microenvironment. The improvement found in overall survival with reduced mTOR expression when CD4+ memory T-cells were enriched was significant, with hazard ratio 2.17, i.e. the group having enriched CD4+ memory T-cells survived at essentially twice the rate. Carcinoma subtypes in this study included all stages, genders, races, grades, mutation burdens, and neoantigen loadings. Expression ranges of the probes were from 437-4839.

An additional study of clinical results for lung squamous cell carcinoma patients was performed which showed improvement in overall survival (OS) with reduced mTOR expression when eosinophils were reduced in a tumor microenvironment. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in lung squamous cell carcinoma and reduced mTOR expression where biomarkers were used to select patients who benefit from the methods and/or agents.

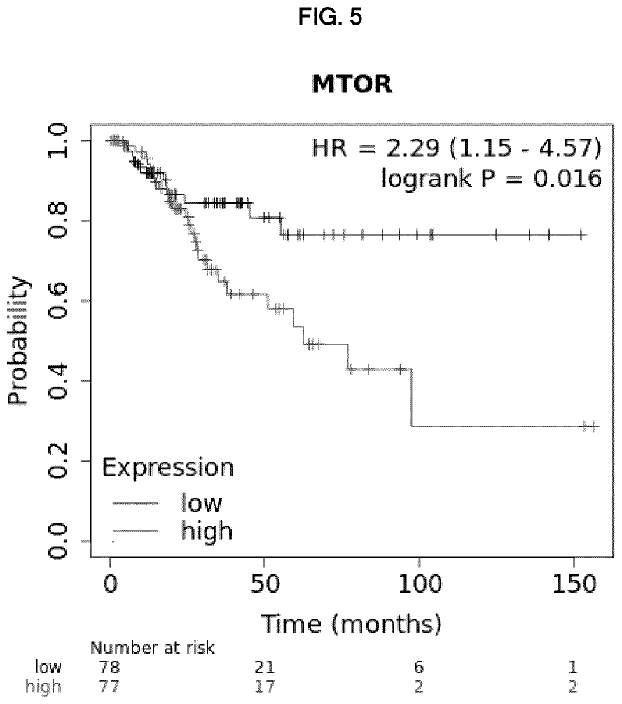

Results of the study for lung squamous cell carcinoma are shown in FIG. 5. FIG. 5 shows highly significant (logrank P=0.0157) improvement in overall survival for patients for which eosinophils were reduced below median, as measured in a tumor microenvironment. The improvement found in overall survival with reduced mTOR expression when eosinophils were reduced was significant, with hazard ratio 2.29, i.e. the group having reduced eosinophils survived at essentially twice the rate. Carcinoma subtypes in this study included all stages, genders, races, grades, mutation burdens, and neoantigen loadings. Expression ranges of the probes were from 437-4839.

Example 3. Methods and agents of this disclosure for suppressing and inhibiting mTOR can be used against pancreatic cancer where biomarkers are used to select patients who benefit from the methods and/or agents. Biomarkers of significance against pancreatic cancer were a level of a CD8+ cell determined in a tumor microenvironment, and a tumor mutation burden (TMB).

A study of clinical results for pancreatic cancer patients was performed which showed improvement in overall survival (OS) with reduced mTOR expression when CD8+ T-cells were enriched in a tumor microenvironment. The improvement found in overall survival with reduced mTOR expression when CD8+ T-cells were enriched for the upper quartile of the study group was a significant increase from 16 months for the high mTOR expression cohort to 24 months for the low mTOR expression cohort. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in pancreatic ductal adenocarcinoma and reduced mTOR expression where biomarkers were used to select patients who benefit from the methods and/or agents.

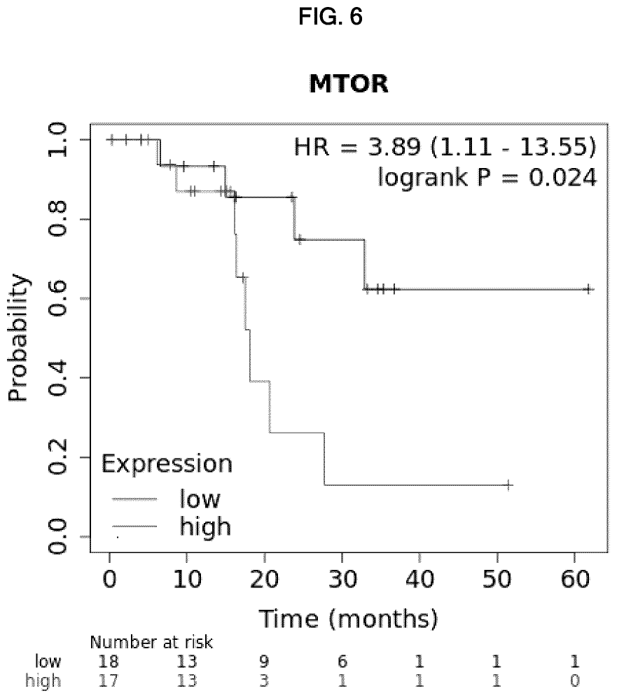

Results of the study for pancreatic ductal adenocarcinoma are shown in FIG. 6. FIG. 6 shows highly significant (logrank P=0.024) improvement in overall survival for patients with reduced mTOR when CD8+ T-cells were enriched above median, as measured in a tumor microenvironment. The improvement found in overall survival with reduced mTOR expression when CD8+ T-cells were enriched was significant, with hazard ratio 3.89, i.e. the group having enriched CD8+ T-cells survived at nearly four-fold increased rate. Cancer subtypes in this study included all stages, genders, races, grades, mutation burdens, and neoantigen loadings. Expression ranges of the probes were from 731-2164.

An additional study of clinical results for pancreatic ductal adenocarcinoma patients was performed which showed improvement in overall survival (OS) with reduced mTOR expression when tumor mutation burden was reduced. The improvement found in overall survival with reduced mTOR expression when tumor mutation burden was reduced for the upper quartile of the study group was a significant increase from 17 months for the high mTOR expression cohort to 35 months for the low mTOR expression cohort. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in pancreatic ductal adenocarcinoma and reduced mTOR expression where biomarkers were used to select patients who benefit from the methods and/or agents.

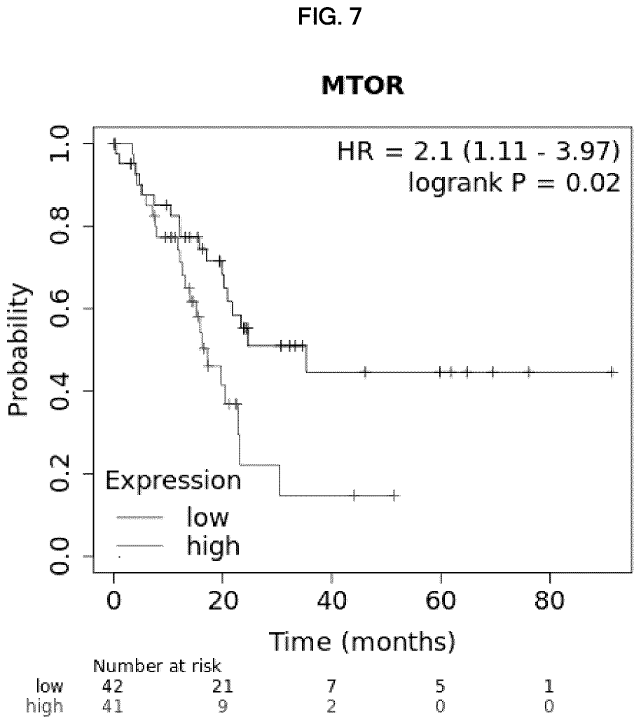

Results of the study for pancreatic ductal adenocarcinoma are shown in FIG. 7. FIG. 7 shows highly significant (logrank P=0.02) improvement in overall survival for patients for which tumor mutation burden was reduced below median. The improvement found in overall survival with reduced mTOR expression when tumor mutation burden was reduced was significant, with hazard ratio 3.97, i.e. the group having reduced tumor mutation burden survived at nearly four-fold increased rate. Cancer subtypes in this study included all stages, genders, races, grades, and neoantigen loadings. Expression ranges of the probes were from 287-2231.

Example 4. Methods and agents of this disclosure for suppressing and inhibiting mTOR can be used against ovarian cancer where biomarkers are used to select patients who benefit from the methods and/or agents. A biomarker of significance against ovarian cancer was a level of neoantigen load.

A study of clinical results for ovarian cancer patients was performed which showed improvement in overall survival (OS) with reduced mTOR expression when a neoantigen load was increased. The improvement found in overall survival with reduced mTOR expression when neoantigen load was increased above median was a significant increase from 40 months for the high mTOR expression cohort to 55 months for the low mTOR expression cohort. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in ovarian cancer and reduced mTOR expression where biomarkers were used to select patients who benefit from the methods and/or agents.

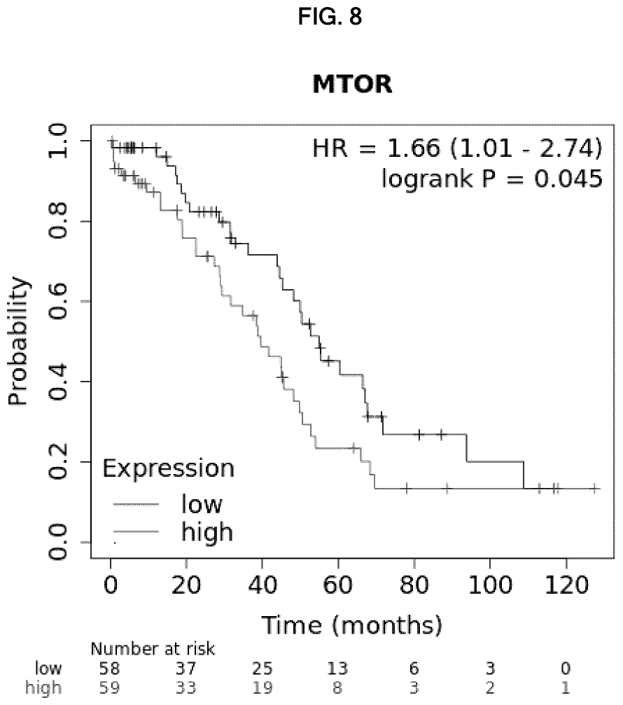

Results of the study for ovarian cancer are shown in FIG. 8. FIG. 8 shows highly significant (logrank P=0.045) improvement in overall survival for patients for which neoantigen load was increased above median. The improvement found in overall survival with reduced mTOR expression when neoantigen load was increased was significant, with hazard ratio 1.66, i.e. the group having reduced neoantigen load survived at nearly 70% increased rate. Cancer subtypes in this study included all stages, genders, races, grades, and mutation burdens. Expression ranges of the probes were from 304-5797. In this study, no restrictions or exclusions were made for tumor microenvironment immune cell components.

Example 5. Methods and agents of this disclosure for suppressing and inhibiting mTOR can be used against sarcoma where biomarkers are used to select patients who benefit from the methods and/or agents. A biomarker of significance against sarcoma was a level of a tumor mutation burden.

A study of clinical results for sarcoma patients was performed which showed improvement in overall survival (OS) with reduced mTOR expression when a tumor mutation burden was reduced. The improvement found in overall survival with reduced mTOR expression when tumor mutation burden was reduced below median for the upper quartile was a significant increase from 23 months for the high mTOR expression cohort to 66 months for the low mTOR expression cohort, a nearly three-fold increase in OS. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in this sarcoma subtype and reduced mTOR expression where biomarkers were used to select patients who benefit from the methods and/or agents.

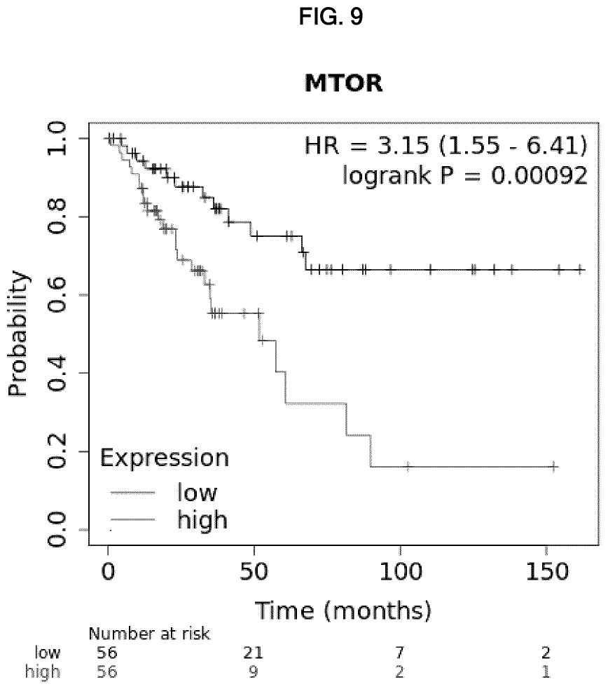

Results of the study for this sarcoma subtype are shown in FIG. 9. FIG. 9 shows highly significant (logrank P=0.00092) improvement in overall survival for patients for which tumor mutation burden was reduced below median. The improvement found in overall survival with reduced mTOR expression when tumor mutation burden was reduced was significant, with hazard ratio 3.15, i.e. the group having reduced tumor mutation burden survived at more than three-fold increased rate. Cancer subtypes in this study included all stages, genders, races, grades, and neoantigen loadings. Expression ranges of the probes were from 429-3103. In this study, no restrictions or exclusions were made for tumor microenvironment immune cell components.

Example 6. Methods and agents of this disclosure for suppressing and inhibiting mTOR can be used against sarcoma where biomarkers are used to select patients who benefit from the methods and/or agents. A biomarker of significance against sarcoma was a level of a neoantigen load.

A study of clinical results for sarcoma patients was performed which showed improvement in overall survival (OS) with reduced mTOR expression when a neoantigen load was reduced. The improvement found in overall survival with reduced mTOR expression when neoantigen load was reduced below median for the upper quartile was a significant increase from 19 months for the high mTOR expression cohort to 36 months for the low mTOR expression cohort, a nearly two-fold increase in OS. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in this sarcoma subtype and reduced mTOR expression where biomarkers were used to select patients who benefit from the methods and/or agents.

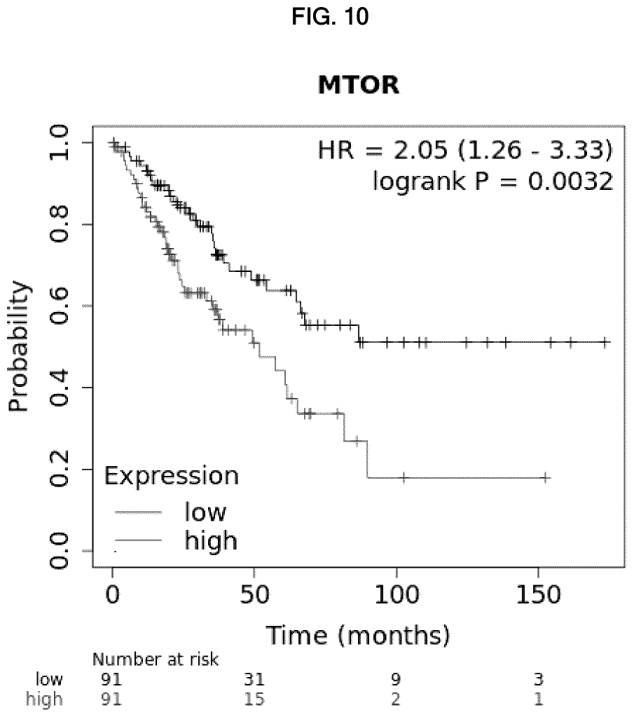

Results of the study for this sarcoma subtype are shown in FIG. 10. FIG. 10 shows highly significant (logrank P=0.0032) improvement in overall survival for patients for which neoantigen load was reduced below median. The improvement found in overall survival with reduced mTOR expression when neoantigen load was reduced was significant, with hazard ratio 2.05, i.e. the group having reduced neoantigen load survived at more than two-fold increased rate. Cancer subtypes in this study included all stages, genders, races, grades, and mutation burden. Expression ranges of the probes were from 345-4010. In this study, no restrictions or exclusions were made for tumor microenvironment immune cell components.

Example 7. Methods and agents of this disclosure for suppressing and inhibiting mTOR can be used against sarcoma where biomarkers are used to select patients who benefit from the methods and/or agents. A biomarker of significance against sarcoma was a level of a mesenchymal stem cell in a tumor microenvironment.

A study of clinical results for sarcoma patients was performed which showed improvement in overall survival (OS) with reduced mTOR expression when a level of a mesenchymal stem cell was increased. The improvement found in median overall survival with reduced mTOR expression when mesenchymal stem cells were increased above median was a significant increase from 39 months for the high mTOR expression cohort to 86 months for the low mTOR expression cohort, a more than two-fold increase in OS. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in this sarcoma subtype and reduced mTOR expression where biomarkers were used to select patients who benefit from the methods and/or agents.

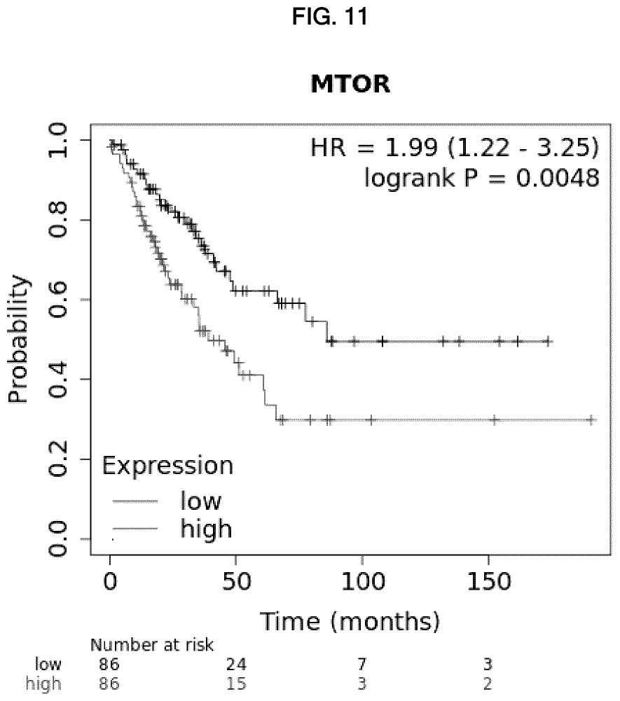

Results of the study for this sarcoma subtype are shown in FIG. 11. FIG. 11 shows highly significant (logrank P=0.0048) improvement in overall survival for patients for which mesenchymal stem cells were increased above median. The improvement found in overall survival with reduced mTOR expression when mesenchymal stem cells were increased was significant, with hazard ratio 1.99, i.e. the group having reduced neoantigen load survived at a two-fold increased rate. Cancer subtypes in this study included all stages, genders, races, grades, mutation burdens, and neoantigen loadings. Expression ranges of the probes were from 345-3147.

Example 8. Methods and agents of this disclosure for suppressing and inhibiting mTOR can be used against sarcoma where biomarkers are used to select patients who benefit from the methods and/or agents. A biomarker of significance against sarcoma was a level of a Type 1 T-helper cell in a tumor microenvironment.

A study of clinical results for sarcoma patients was performed which showed improvement in overall survival (OS) with reduced mTOR expression when a level of a Type 1 T-helper cell was increased. The improvement found in median overall survival with reduced mTOR expression when Type 1 T-helper cells were increased above median was a significant increase from 28 months for the high mTOR expression cohort to 86 months for the low mTOR expression cohort, a more than three-fold increase in OS. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in this sarcoma subtype and reduced mTOR expression where biomarkers were used to select patients who benefit from the methods and/or agents.

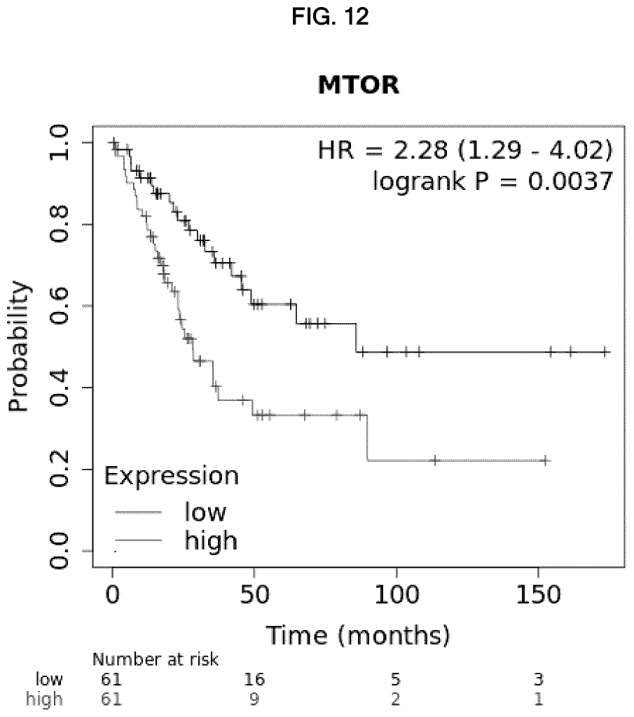

Results of the study for this sarcoma subtype are shown in FIG. 12. FIG. 12 shows highly significant (logrank P=0.0037) improvement in overall survival for patients for which Type 1 T-helper cells were increased above median. The improvement found in median overall survival with reduced mTOR expression when Type 1 T-helper cells were increased was significant, with hazard ratio 2.28, i.e. the group having increased Type 1 T-helper cells survived at a more than two-fold increased rate. Cancer subtypes in this study included all stages, genders, races, grades, mutation burdens, and neoantigen loadings. Expression ranges of the probes were from 345-3522.

Example 9. Methods and agents of this disclosure for suppressing and inhibiting mTOR can be used against sarcoma where biomarkers are used to select patients who benefit from the methods and/or agents. Biomarkers of significance against sarcoma were a level of a Type 1 T-helper cell in a tumor microenvironment and a level of a mesenchymal stem cell in a tumor microenvironment.

A study of clinical results for sarcoma patients was performed which showed improvement in overall survival (OS) with reduced mTOR expression when a level of a Type 1 T-helper cell and a level of a mesenchymal stem cell were increased. The improvement found in median overall survival with reduced mTOR expression when Type 1 T-helper cells and mesenchymal stem cells were increased above median was a significant increase from 12 months for the high mTOR expression cohort to 33 months for the low mTOR expression cohort, a nearly three-fold increase in OS. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in this sarcoma subtype and reduced mTOR expression where biomarkers were used to select patients who benefit from the methods and/or agents.

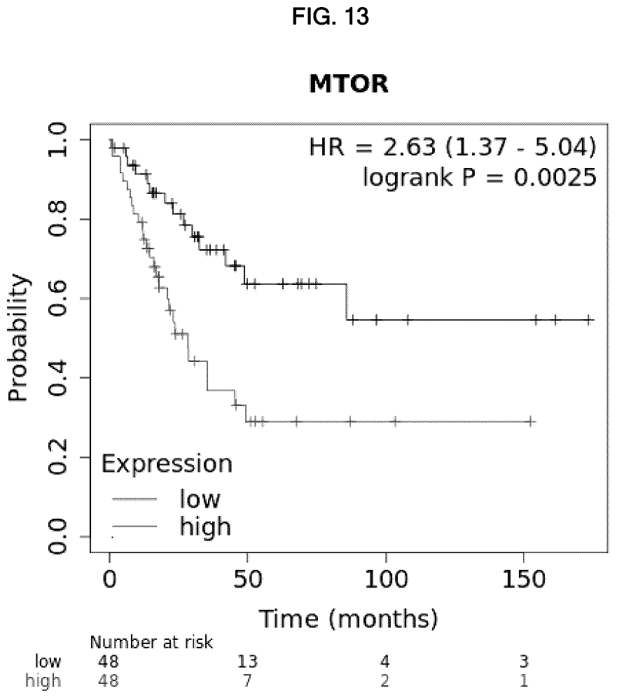

Results of the study for this sarcoma subtype are shown in FIG. 13. FIG. 13 shows highly significant (logrank P=0.0025) improvement in overall survival for patients for which Type 1 T-helper cells and mesenchymal stem cells were increased above median. The improvement found in median overall survival with reduced mTOR expression when Type 1 T-helper cells and mesenchymal stem cells were increased was significant, with hazard ratio 2.63, i.e. the group having increased Type 1 T-helper cells and mesenchymal stem cells survived at a more than two-fold increased rate. Cancer subtypes in this study included all stages, genders, races, grades, mutation burdens, and neoantigen loadings. Expression ranges of the probes were from 345-3103.

Example 10. Methods and agents of this disclosure for suppressing and inhibiting mTOR can be used against sarcoma where biomarkers are used to select patients who benefit from the methods and/or agents. Biomarkers of significance against sarcoma were a level of a Type 1 T-helper cell in a tumor microenvironment, a level of a mesenchymal stem cell in a tumor microenvironment, and tumor mutation burden.

A study of clinical results for sarcoma patients was performed which showed improvement in overall survival (OS) with reduced mTOR expression when tumor mutation burden was reduced, a level of a Type 1 T-helper cell was increased, and a level of a mesenchymal stem cell was increased. The improvement found in median overall survival with reduced mTOR expression when Type 1 T-helper cells and mesenchymal stem cells were increased above median, while tumor mutation burden was reduced, was a significant increase from 12 months for the high mTOR expression cohort to 49 months for the low mTOR expression cohort, a more than four-fold increase in OS. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in this sarcoma subtype and reduced mTOR expression where biomarkers were used to select patients who benefit from the methods and/or agents.

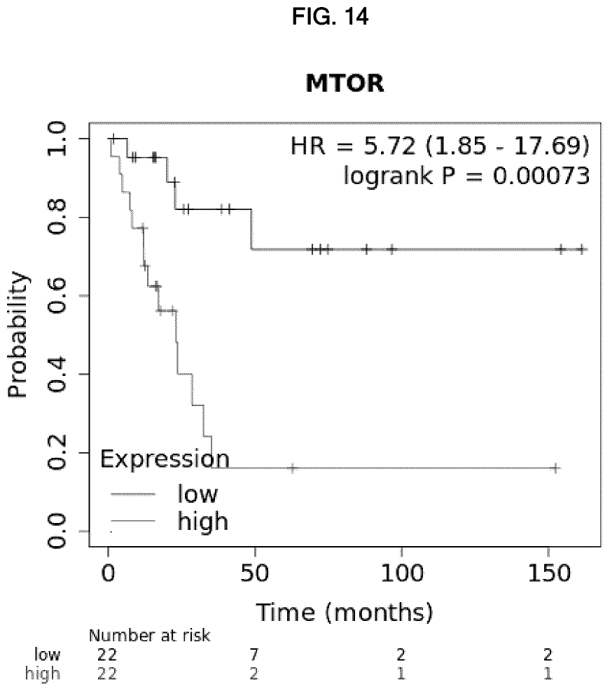

Results of the study for this sarcoma subtype are shown in FIG. 14. FIG. 14 shows highly significant (logrank P=0.00073) improvement in overall survival for patients for which Type 1 T-helper cells and mesenchymal stem cells were increased above median, while tumor mutation burden was reduced below median. The improvement found in median overall survival with reduced mTOR expression when Type 1 T-helper cells and mesenchymal stem cells were increased, while tumor mutation burden was reduced, was significant, with hazard ratio 5.73, i.e. the group having increased Type 1 T-helper cells and mesenchymal stem cells and reduced tumor mutation burden survived at an almost six-fold increased rate. Cancer subtypes in this study included all stages, genders, races, grades, and neoantigen loadings. Expression ranges of the probes were from 429-3103.

Example 11. Methods and agents of this disclosure for suppressing and inhibiting mTOR can be used against sarcoma where biomarkers are used to select patients who benefit from the methods and/or agents. Biomarkers of significance against sarcoma were a level of a Type 1 T-helper cell in a tumor microenvironment and a neoantigen load.

A study of clinical results for sarcoma patients was performed which showed improvement in overall survival (OS) with reduced mTOR expression when neoantigen load was reduced and a level of a Type 1 T-helper cell was increased. The improvement found in median overall survival with reduced mTOR expression when Type 1 T-helper cells were increased above median, while neoantigen load was reduced, was a significant increase from 12 months for the high mTOR expression cohort to 30 months for the low mTOR expression cohort, a more than two-fold increase in OS. This was a highly surprising result because data from a total of over 900 other cancer patients having a wide range of different cancers did not show such improvement with reduced mTOR expression without selecting patients using biomarkers.

Kaplan-Meier Plotter was used to calculate patient survival to determine the connection between improved overall survival in this sarcoma subtype and reduced mTOR expression where biomarkers were used to select patients who benefit from the methods and/or agents.

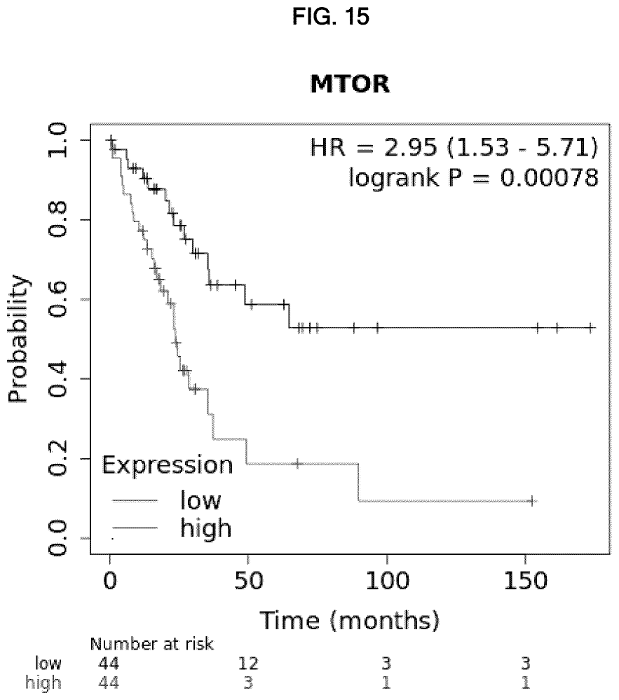

Results of the study for this sarcoma subtype are shown in FIG. 15. FIG. 15 shows highly significant (logrank P=0.00078) improvement in overall survival for patients for which Type 1 T-helper cells were increased above median, while neoantigen load was reduced below median. The improvement found in median overall survival with reduced mTOR expression when Type 1 T-helper cells were increased, while neoantigen load was reduced, was significant, with hazard ratio 2.95, i.e. the group having increased Type 1 T-helper cells and reduced neoantigen load survived at an almost three-fold increased rate. Cancer subtypes in this study included all stages, genders, races, grades, and mutation burdens. Expression ranges of the probes were from 345-3522.

1-24. (canceled)

25. A method for treating or ameliorating a symptom of cancer in a human or animal subject in need, the method comprising:
administering a therapeutically sufficient amount of a pharmaceutical composition comprising an mTOR-specific antisense oligonucleotide in combination with an agent for inhibiting mTOR to the subject.

26. The method of claim 25, wherein the cancer is a sarcoma, a lung cancer, an ovarian cancer, or a pancreatic cancer.

27. The method of claim 25, comprising using one or more biomarkers to select subjects who benefit from the method.

28. The method of claim 27, wherein the one or more biomarkers are a level of a tumor mutation burden (TMB), a tumor neoantigen, a tumor-associated immune cell, or a combination thereof.

29. The method of claim 27, wherein the one or more biomarkers are a level of a basophil cell determined in a tumor microenvironment, a B-cell determined in a tumor microenvironment, a T-cell determined in a tumor microenvironment, a T-helper cell (Th) determined in a tumor microenvironment, an eosinophil cell determined in a tumor microenvironment, a macrophage cell determined in a tumor microenvironment, a mesenchymal stem cell determined in a tumor microenvironment, or a combination thereof.

30. The method of claim 27, wherein the one or more biomarkers are a level of a CD4+ cell determined in a tumor microenvironment, a memory T-cell determined in a tumor microenvironment, a CD8+ cell determined in a tumor microenvironment, a natural killer T-cell determined in a tumor microenvironment, a regulatory T-cell determined in a tumor microenvironment, a type 1 T-helper cell (Th1) determined in a tumor microenvironment, a type 2 T-helper cell (Th2) determined in a tumor microenvironment, or a combination thereof.

31. The method of claim 27, wherein the cancer is a sarcoma and the one or more biomarkers are a level of a tumor mutation burden (TMB), a tumor neoantigen level, a mesenchymal stem cell determined in a tumor microenvironment, a type 1 T-helper cell (Th) determined in a tumor microenvironment, or a combination thereof.

32. The method of claim 27, wherein the cancer is a lung squamous cell carcinoma and the one or more biomarkers are a level of a CD4+ cell determined in a tumor microenvironment, a memory T-cell determined in a tumor microenvironment, an eosinophil cell determined in a tumor microenvironment, or a combination thereof.

33. The method of claim 27, wherein the cancer is a pancreatic cancer and the one or more biomarkers are a level of a CD8+ cell determined in a tumor microenvironment, a tumor mutation burden (TMB), or a combination thereof.

34. The method of claim 27, wherein the cancer is an ovarian cancer and the one or more biomarkers are a level of a natural killer T-cell determined in a tumor microenvironment, a tumor neoantigen determined in a tumor microenvironment, or a combination thereof.

35. The method of claim 25, wherein the mTOR-specific antisense oligonucleotide is complementary to a mTOR transcript and 15-30 nucleotides in length, or complementary to a mTOR pre-RNA, pre-mRNA or mRNA and 18-21 nucleotides in length.

36. The method of claim 25, wherein the mTOR-specific antisense oligonucleotide comprises any of the 20 nucleotide length sequences in the following table:

| SEQ ID | | ANTISENSE SEQUENCE |
|---|---|---|
| NO: | Ref. | 5′→3” |
| 1 | 719-738 | TTCGGCTCACGCTGGGTTGT |
| 2 | 974-993 | CGAGGTTTTGTTCCGAAGCC |
| 3 | 985-1004 | GGGTAATGTGACGAGGTTTT |
| 4 | 986-1005 | GGGGTAATGTGACGAGGTTT |
| 5 | 1163-1182 | CACACCTGATCAAATTTCTC |
| 6 | 1164-1183 | GCACACCTGATCAAATTTCT |
| 7 | 2670-2689 | CTGCTCAGTCTTCAGAAAAT |
| 8 | 2671-2690 | TCTGCTCAGTCTTCAGAAAA |
| 9 | 4121-4140 | AAGTTTAAGAGGGTCTGTGT |
| 10 | 5115-5134 | TGTTGGCAGAGGATGGTCAA |
| 11 | 5117-5136 | ACTGTTGGCAGAGGATGGTC |
| 12 | 5355-5374 | TTTGGGGATTGTGCTCTCAT |
| 13 | 5467-5486 | GGTTCTGATGTTTGTAGTGT |
| 14 | 5468-5487 | TGGTTCTGATGTTTGTAGTG |
| 15 | 5473-5492 | GGGCTTGGTTCTGATGTTTG |
| 16 | 5474-5493 | CGGGCTTGGTTCTGATGTTT |
| 17 | 6481-6500 | TGGGGTCATATGTTCCTGGC |
| 18 | 6482-6501 | TTGGGGTCATATGTTCCTGG |
| 19 | 7769-7788 | GGTTTCAGTTTAGTGGAAGC |
| 20 | 8297-8316 | TGAATTGAAGCGTGTGAGTC |

and chemically-modified variants thereof, LNA variants thereof, gapmer variants thereof, and pooling and combinations thereof.

37. The method of claim 36, wherein the mTOR-specific antisense oligonucleotides have no more than one or two mismatches as compared to a target human mTOR.

38. The method of claim 36, wherein the mTOR-specific antisense oligonucleotides reduce a mTOR transcript level by at least 60%, or at least 70%, or at least 80%, or at least 90%.

39. The method of claim 36, wherein the mTOR-specific antisense oligonucleotides have one or more nucleotides chemically modified as a phosphorothioate internucleoside linkage, a methoxypropylphosphonate internucleoside linkage, an aminophosphoro linkage to a morpholino group, a 2′-OMe ribose group, a 2′-MOE methoxyethyl ribose group, a 2′-4′ constrained methoxyethyl bicyclic ribose group, a 2′-4′ constrained ethyl bicyclic ribose group, an LNA ribose group, a 2′-F ribose group, or a 5-methylcytodine base.

40. The method of claim 36, wherein the antisense oligonucleotide is conjugated to a polyethylene glycol, a lipid, or a triantennary N-acetyl-galactosamine.

41. The method of claim 36, comprising antisense oligonucleotides in a carrier of sterile water for injection, saline, isotonic saline, or a combination thereof, and wherein the antisense oligonucleotide is stable for at least 14 days in carrier at 37° C.

42. The method of claim 36, comprising antisense oligonucleotides substantially free of excipients.

43. The method of claim 25, wherein the agent for inhibiting mTOR is rapamycin, everolimus, temsirolimus, sirolimus, deforolimus, ridaforolimus, zotarolimus, torkinib, samotolisib, omipalisib, apitolisib, vistusertib, dactolisib, gedatolisib, voxtalisib, chrysophanic, or a combination thereof.

44. The method of claim 25, wherein the agent for inhibiting mTOR is 1-[4-[4-(1-Oxopropyl)-1-piperazinyl]-3-(trifluoromethyl)phenyl]-9-(3-quinolinyl)-benzo[h]-1,6-naphthyridin-2(1H)-one, 3-[4-(4-Morpholinylpyrido[3′,2′:4,5]furo[3,2-d]pyrimidin-2-yl]phenol hydrochloride, N-[4-[4-(4-Morpholinyl)-1-[1-(3-pyridinylmethyl)-4-piperidinyl]-1H-pyrazolo[3,4-d]pyrimidin-6-yl]phenyl]-carbamic acid methyl ester dihydrochloride, 2,4-Difluoro-N-[2-methoxy-5-[4-(4-pyridazinyl)-6-quinolinyl]-3-pyridinyl]benzenesulfonamide (omipalisib), 5-Chloro-N-(2-chloro-4-nitrophenyl)-2-hydroxybenzamide (niclosamide), or a combination thereof.

45. The method of claim 25, wherein the administration is combined with a standard of care treatment for the cancer, wherein the standard of care treatment comprises chemotherapy or radiation therapy, and wherein the administration is performed in the absence of immune checkpoint inhibitors.

46. The method of claim 25, wherein the mTOR-specific antisense oligonucleotide and agent for inhibiting mTOR are each administered concurrently, simultaneously, sequentially, or separately in time.

47. (canceled)

48. The method of claim 25, wherein the administration increases survival rate at month 6, 12, 18, 24, 30, or 36.
